(19) United States
(12) Patent Application Publication
(10) Pub. No.: US 2011/0268770 A1
(43) Pub. Date: Nov. 3, 2011

(54) CHEWABLE GELLED EMULSIONS

(75) Inventors: Tore Seternes, Tromso (NO); Kurt Ingar Draget, Tromso (NO); Ingvild Johanne Haug, Tromso (NO)

(73) Assignee: PROBIO ASA, Tromso (NO)

(21) Appl. No.: 13/123,163

(22) PCT Filed: Oct. 8, 2009
(86) PCT No.: PCT/GB2009/002406

§ 371 Date: Jul. 11, 2011

(30) Foreign Application Priority Data
Oct. 8, 2008 (GB) 0818472.3
May 12, 2009 (GB) 0908157.1

(51) Int. Cl. A61K 31/405; A61K 31/40; B65D 83/04; A61P 3/06; A61P 9/00; A61K 31/351; A61K 9/107

(52) U.S. Cl. 424/400; 514/460; 514/419; 514/423; 206/532

(57) ABSTRACT

An oral pharmaceutical composition in unit dose form, each unit dose comprising a statin within a unitary carrier body, said body comprising a soft, chewable, gelled oil-in-water emulsion, one or both of the oil phase and the water phase whereof comprises a physiologically tolerable omega-3 acid ester.

This invention relates to oral pharmaceutical compositions comprising a soft, gelled oil-in-water emulsion containing a statin and a physiologically tolerable ester of an omega-3 fatty acid, to processes for their preparation, and to their use.

Statins, hydroxy-methylglutaryl coenzyme A (HMG-CoA) reductase inhibitors, are increasingly being used to treat patients with cardiovascular problems to reduce blood cholesterol levels. Examples of typical statins include mevastatin, simvastatin (Zocor®), lovastatin (Mevacor®), fluvastatin (Lescol®), pravastatin (Pravacol®), atorvastatin (Lipitor®), cerivastatin (Baycol®), Pitavastatin®), and rosuvastatin (Crestor®).

It was reported in 2007 that combination therapy with both a statin and an omega-3 acid ester offers particular benefit (see Yokoyama et al, Lancet 369:1090-1098 (2007)) and currently this combined therapy is being widely adopted. Such combined therapies have also been proposed by Reliant Pharmaceuticals Inc. in WO2006/096806, US-A-2007/0191467 and WO2008/045170, the contents of which are hereby incorporated by reference.

Yokoyama et al reported the results of a major analysis and showed a reduction in mortality from coronary heart disease. 9326 patients received statin plus 1800 mg daily of eicosapentaenoic acid (EPA), while 9319 patients received statin alone. The mortality rate from combined therapy was reduced by 19%. Statins were administered at 10 or 20 mg daily of pravastatin or 5 or 10 mg daily of simvastatin. The EPA was administered as two capsules each containing 300 mg ethyl EPA ester, taken three times per day.

In WO2006/096806, Reliant (which in December 2007 was acquired by GlaxoSmithKline (see http://www.pharmiweb.com/PressReleases/pressrel.asp?ROW_ID=3003) disclosed pharmaceutical compositions in the form of statins dissolved in omega-3 acid esters. The preferred daily omega-3 dose was stated to be 0.75 to 4 g. The statins used included simvastatin, atorvastatin and fluvastatin, although the use of other stains was suggested. The omega-3 acid ester used in most of the examples was K85EE, a material available from Pronova Biocare AS, Lysaker, Norway. K85EE, also known as Omacor® is a 90% mixture of ethyl EPA ester and ethyl DHA ester. Pronova Biocare market the regulatory-approved product Omacor®which is also marketed by GlaxoSmithKline as Lovaza®. Lovaza® is marketed as a lipid-regulating agent as 1-gram gel capsules containing about 475 mg of ethyl EPA ester and about 375 mg of ethyl DHA ester. The recommended dosage four capsules per day, taken together or as two capsules twice a day, i.e. a total omega-3 ester dose of about 3600 mg.

In US-A-2007/0191467 and WO2008/045170, Reliant report beneficial results of combined therapy with Omacor® and simvastatin at 4 g/day Omacor® and 40 mg/day simvastatin.

Polyunsaturated fatty acid esters such as omega-3 esters are very oxidation sensitive and routinely are administered as soft gelatin capsules containing a liquid ester core. This is the conventional administration form of for example fish oils and evening primrose oil which are sold as nutraceuticals containing omega-3.

Thus the combined statin/omega-3 ester therapy for combating cardiovascular disease involves the patient, who may well be elderly, consuming a small amount of statin and a large amount of omega-3 ester, the omega-3 ester being administered in a large number of small capsules or as a smaller number of large capsules (a 1 g capsule is large).

When the unit dose of a drug substance is large, the oral unit dosage forms, e.g. tablets or capsules, may likewise be large and so difficult for elderly or young patients to swallow and moreover may cause a gagging reaction even with healthy adults. Accordingly, any therapeutic or prophylactic dosage regime which involves the consumption of large numbers of dose units or numbers of large, difficult to swallow, dose units is inherently at risk of patient non-compliance.

However, we have now found that the statin/omega-3 combination may be administered without these problems when presented as a piece of soft, chewable, gelled oil-in-water emulsion.

Moreover, we have found that the uptake of lipophilic compounds is increased by providing such compounds in the form of a soft, gelled oil-in-water emulsion.

Thus viewed from one aspect the invention provides an oral pharmaceutical composition in unit dose form, each unit dose comprising a statin within a unitary carrier body, said body comprising a soft, chewable, gelled oil-in-water emulsion, one or both of the oil phase and the water phase whereof comprises a physiologically tolerable omega-3 acid ester, particularly an EPA ester, a docosahexaenoic acid (DHA) ester or a combination of EPA and DHA esters.

In a particularly preferred embodiment, the statin is present in the aqueous phase of the gelled emulsion, e.g. in dissolved or dispersed, e.g. microencapsulated, form. Microencapsulation in this regard may be achieved by coating the statin with a hydrophilic substance, for example by mixing with an amphiphilic agent and then dispersion in an aqueous medium to form vesicles containing the statin and having a hydrophilic outer surface. Liposome-encapsulation of statins is described for example in U.S. Pat. No. 5,004,611, the disclosures of which are hereby incorporated by reference. Likewise, lipid-soluble statins may be dissolved in an oil which is essentially omega-3 free, e.g. a saturated or unsaturated fatty acid phospholipid, and then emulsified in an aqueous solution of the gelling agent to form a dispersion of statin-containing vesicles, whereafter the omega-3 ester may be added, an emulsion formed, and the aqueous phase allowed to gel.

The commercially available statins tend to fall into two classes—those which are water-soluble (such as pravastatin and rosuvastatin), and those which are lipid-soluble (such as lovastatin and simvastatin). The water-soluble statins are readily presented in the aqueous phase of the gelled emulsion in dissolved form, while the lipid-soluble statins may be constrained to remain in the aqueous phase, for example by microencapsulation as discussed above. In general, it is thought that the use of water-soluble statins may be more beneficial to the patient as well as more simple to formulate in the aqueous phase of the gelled emulsion. The decreasing order of lipophilicity for some of the major statins appears to be lovastatin, simvastatin, atorvastatin, fluvastatin, rosuvastatin and pravastatin.

Viewed from a further aspect, the invention provides a process for preparing an oral pharmaceutical composition in unit dose form, which process comprises: forming an oil phase comprising a physiologically tolerable oil which comprises a physiologically tolerable omega-3 acid ester; forming an aqueous phase comprising an aqueous solution of a physiologically tolerable gelling agent; forming an oil-in-water emulsion with said oil phase and said aqueous phase; allowing said emulsion to gel to form a soft chewable mass; and, before, during or after gelling of said emulsion, dividing said emulsion into dose units; wherein a statin is incorporated within one or both of said oil and water phases, preferably the oil phase.

Viewed from a further aspect, the invention provides a process for preparing an oral pharmaceutical composition in unit dose form, which process comprises: forming an oil phase comprising a physiologically tolerable oil (e.g. a polyunsaturated fatty acid ester, such as an omega-3 acid ester, an omega-6 acid ester, an omega-9 acid ester or a vegetable oil, preferably an omega-3 acid ester, especially an EPA ester, a docosahexaenoic acid (DHA) ester or a combination of EPA and DHA esters); forming an aqueous phase comprising an aqueous solution of a statin dissolved or dispersed therein; forming a water-in-oil emulsion with said oil phase and said aqueous phase, forming an oil-in-water emulsion with said water-in-oil phase and a further aqueous phase, comprising an aqueous solution of a physiologically tolerable gelling agent, allowing said emulsion to gel to form a soft chewable mass; and, before, during or after gelling of said emulsion, dividing said emulsion into dose units.

The soft, gelled dose units of the present invention can remain intact during passage through the stomach and release the drug substances disposed within the gel matrix further down the gastrointestinal tract where the environment is not so harsh and where uptake is feasible. In this format some of the drug substance at the periphery of the matrix may be degraded by gastric fluid during stomach transit. Nonetheless, the soft, gelled dose units of the present invention have the advantage of being chewable and so more easily swallowed if large, e.g. above 1000 mg, more especially 1500 to 5000 mg. In the case where a large dose is required, the advantage of a single chewable dose unit may outweigh the relatively small loss of drug substance from the periphery of the chewed fragments during stomach transit. Chewable gel units moreover have the advantage that patient compliance is greater for patients with a gag reaction to swallowing tablets or capsules intact, in particular juvenile or elderly patients.

Thus viewed from one aspect the invention provides an oral pharmaceutical composition in unit dose form, each unit dose comprising a statin within a unitary carrier body, said body comprising a soft, chewable, gelled oil-in-water emulsion, capable of remaining substantially intact during passage through the stomach, one or both of the oil phase and the water phase whereof comprises a physiologically tolerable omega-3 acid ester, particularly an EPA ester, a docosahexaenoic acid (DHA) ester or a combination of EPA and DHA esters.

By soft and chewable it is meant that the gelled emulsion is preferably readily deformable rather than rigid while yet being self supporting, i.e. that it will not flow like a viscous liquid, and that it may be readily fragmented upon chewing, i.e. so that it need not be swallowed whole. Typically, such a gelled emulsion may be compressed at least substantially reversibly, i.e. elastically, by at least 10%, preferably at least 40% upon application of a force/deformation gradient of 0.1 mm/s at 21° C., 50% relative humidity and atmospheric pressure.

Preferably the compression breaking strengths of the soft, gelled dose units of the present invention are greater than 500 g/cm², particularly greater than 1000 g/cm², especially preferably greater than 2000 g/cm², e.g. 2900-3600 g/cm².

By unitary carrier body, it is meant that each dose unit contains one piece of gelled emulsion. Such pieces may be referred to hereinafter as “cores”.

The cores may be formed from larger pieces of gelled emulsion, e.g. by cutting, or, more preferably by extrusion or molding of dose units of incompletely gelled emulsion.

By statin is meant an HMG CoA-reductase inhibitor, preferably one with regulatory approval. The use of cerivastatin, which has been withdrawn from the market is not preferred. The use of lovastatin, simvastatin, pravastatin, atorvastatin, and rosuvastatin are especially preferred.

The quantity of statin per unit dose of the compositions of the invention will conveniently be in the range of 10 to 150%, especially 20 to 100%, of the normal recommended daily adult or child dose. Such dosages may readily be ascertained from the manufacturers' publications and websites. Typically, the daily dosage will be in the range 1 to 100 mg, especially 2 to 80 mg.

Preferred statin contents per dose unit are about 5 to 100 mg, e.g. about 5, 10, 20, 40 or 80 mg for simvastatin, about 20, 40 or 60 mg for lovastatin, about 20, 40 or 80 mg for fluvastatin, about 10, 20, 40 or 80 mg for pravastatin, about 10, 20, 40 or 80 mg for atorvastatin, and about 5, 10, 20 or 40 mg for rosuvastatin.

The compositions of the invention especially preferably consist of cores of gelled emulsion. However, less preferably, they may comprise a gelled emulsion core provided with a coating of a physiologically tolerable coating material. Such coatings may be of the type conventional within the pharmaceutical industry and may be applied by conventional means, e.g. spraying or dipping. For some applications, especially pediatric applications, a thin sugar (or otherwise sweetened) coating may be desired. Unless it is rapidly soluble in the mouth, however, rigid coatings are generally not desired since it is central to the invention that the soft gelled core be chewable so as to facilitate swallowing.

It is preferred that the cores be non-spherical as this facilitates chewing. While disc and lenticular forms are suitable, it is preferred that the cores be elongate, for example having cylindrical or similar form (optionally of course with rounded ends and one or more planar side faces). Where the application is pediatric, the cores may be in child-attractive forms, e.g. in a geometric shape or in the shape of an animal or cartoon character. In this way, the unit dose may be consumed with ease by patients who otherwise might have difficulty swallowing a conventional tablet or capsule, e.g. the young, the old, those with gag reactions, patients on chemotherapy, and others with reduced mouth function.

Since one major benefit of the compositions of the invention lies in their ease of consumption relative to conventional tablets or capsules, the cores will generally be quite large, e.g. having a mass of 100 to 3000 mg, especially 400 to 2000 mg, particularly 600 to 1500 mg.

The oil of the oil phase in the compositions of the invention may be any physiologically tolerable omega-3 containing oil, but will preferably be one or a mixture of fatty acid esters (for example phospholipids, mono-, di- or tri-glycerides, and lower alkyl esters). Such materials may be natural, synthetic or semi-synthetic. The use of plant and marine oils (e.g. oils from plant seeds, algae, fish (especially oily fish), microorganisms and marine invertebrates (especially krill)) is especially preferred, as is the use of DHA and/or EPA C₁₋₆ alkyl, especially ethyl, esters.

Typically the oil phase will constitute 0.05 to 5 g, preferably 0.1 to 3 g, especially 0.2 to 2 g, particularly 0.3 to 1.25 g, more particularly 0.4 to 1.0 g, per dose unit. Alternatively put, the oil phase preferably constitutes 5 to 75% wt., especially 30 to 50% wt., e.g. 40 to 50% wt. of the dose unit. The omega-3 acid ester content per dose unit, expressed as weight of free omega-3 acid per gram oil phase will typically be 10 to 90%, especially 30 to 85% wt. The DHA and/or EPA content is likewise preferably within these ranges as these are the preferred omega-3 acids.

The gelling agent used in the aqueous phase of the emulsion may be any physiologically tolerable gelling agent (preferably a saccharide (e.g. an oligosaccharide or polysaccharide), a protein or a glycoprotein) or combination capable of forming a soft, chewable, self-supporting gelled oil-in-water emulsion. Many such materials are known from the food and pharmaceutical industry and are discussed for example in Handbook of hydrocolloids, G O Phillips and P A Williams (Eds.), Woodhead Publishing, Cambridge, UK, 2000. The gelling agents are preferably materials capable of undergoing a sol-gel transformation, e.g. under the influence of a change in physiochemical parameters such as temperature, pH, presence of metal ions (e.g. group 1 or 2 metal ions), etc. Preferred gelling agents include gelatins, alginates and carageenans. However, the use of gelatins is especially preferred as breakdown in the throat of trapped fragments is ensured and as cores having the desired properties may readily be produced using gelatins.

Here it should be emphasized that the gelled emulsion should be self-supporting, soft and fragmentable on chewing. It is not desired that the gelled emulsion should dissolve rapidly in the mouth without chewing as the administration of the composition would then differ little functionally from administration of an oil solution of the statin. Gelatin can be used to give the gelled emulsions these desired characteristics.

The gelatins used as gelling agents in the composition of the invention may be produced from the collagen of any mammal or the collagen of any aquatic species, however the use of gelatin from salt-water fish and in particular cold and warm water fishes is preferred.

Gelatins having an imino acid content of 5 to 25% wt. are preferred, more especially those having an imino acid content of 10 to 25% wt. The gelatins will typically have a weight average molecular weight in the range 10 to 250 kDa, preferably 75 to 220 kDa, especially 80 to 200 kDa. Gelatins having no Bloom value or low Bloom values of 60-300, especially 90-200 are preferred. Where a gelatin of no Bloom value, e.g. a cold water fish gelatin, is used, this will typically be used together with another gelatin or other gelling agent. The combination of cold water and warm water fish gelatins is especially preferred. The gelatin will typically be present in the aqueous phase at a concentration of 1 to 50% wt., preferably 2 to 35% wt., particularly 5 to 25% wt. In the case of mixtures of gelatin and polysaccharides, the weight ratio of gelatin to polysaccharide in the aqueous phase will typically be 50:1 to 5:1, preferably 40:1 to 9:1, especially 20:1 to 10:1.

Where polysaccharides, or mixtures of polysaccharides and gelatin are used as the gelling agent, it is preferred to use natural polysaccharides, synthetic polysaccharides or semisynthetic polysaccharides, e.g. polysaccharides from plants, fish, terrestrial mammals, algae, bacteria and derivatives and fragmentation products thereof. Typical marine polysaccharides include carageenans, alginates, agars and chitosans.

Typical plant polysaccharides include pectins. Typical microorganism polysaccharides include gellans and scleroglucans. The use of charged, e.g. electrostatically charged and/or sulphated polysaccharides is preferred, as is the use of marine polysaccharides, in particular carageenans, and alginates, especially carageenans. Carageenans are used below as representative polysaccharide gelling agents.

The carageenan family, which includes iota- and kappa-carageenans, is a family of linear sulphated polysaccharides produced from red algae. The repeating disaccharide unit in kappa-carrageenan is β-D-galactose-4-sulphate and 3,6-anhydro-α-D-galactose, while that in iota-carrageenan is β-D-galactose-4-sulphate and 3,6-anhydro-α-D-galactose-2-sulphate. Both kappa- and iota-carageenans are used in food preparations. The carageenans are used as stabilisers, emulsifiers, gelling agents and fat replacers.

Both iota and kappa carageenans form salt- or cold-setting reversible gels in an aqueous environment. Coil-helix transition and aggregation of helices form the gel network. Kappa-carrageenan has binding sites for specific monovalent cations, resulting in gel formation with decreasing shear and elastic moduli in the order Cs⁺>K⁺>>Na⁺>Li⁺. As a rule, an increasing salt concentration enhances the elastic modulus and the setting and melting temperatures of a kappa-carrageenan gel. The use of water-soluble potassium, rubidium, or caesium compounds, particularly potassium compounds, and particularly naturally occurring compounds (e.g. salts) is preferred when kappa-carrageenan is used according to the invention, e.g. at concentrations of up to 100 mM, more especially up to 50 mM. A salt-dependent conformational transition is also found for iota-carrageenan. The molecules are also known to undergo coil-helix transition with strong helix-stabilisation in the presence of multivalent cations, like Ca²⁺. The use of water-soluble calcium, strontium, barium, iron or aluminium compounds, especially calcium compounds, and particularly naturally occurring compounds (e.g. salts) is preferred when iota-carrageenan is used according to the invention, e.g. at concentrations of up to 100 mM.

The polysaccharide gelling agents used according to the invention will typically have weight average molecular weights of 5 kDa to 2 MDa, preferably 10 kDa to 1 MDa, most preferably 100 kDa to 900 kDa, particularly 200 to 800 kDa. They will typically be used at concentrations of 0.01 to 5% wt, preferably 0.1 to 1.5% wt., particularly 0.2 to 1% wt in the aqueous phase. Where mono or multivalent cations, typically group 1 or group 2 metal ions, are included in the aqueous phase, this will typically be at concentrations in the range 2.5 to 100 mM, particularly 5 to 50 mM.

Besides the gelling agent and water and any required gelling initiator, other physiologically tolerable materials may be present in the aqueous phase, e.g. emulsifiers, emulsion stabilizers, pH modifiers, viscosity modifiers, sweeteners, fillers, vitamins (e.g. vitamin C, thiamine, riboflavin, niacin, vitamin B6, vitamin B12, folacin, panthotenic acid), minerals, aromas, flavours, colours, physiologically active agents, etc. It is especially preferred that a lipophilic antioxidant, e.g. vitamin E, be included in the oil phase. Other vitamins which may be present in the oil phase are vitamin A, vitamin D and vitamin K. Vitamins may classify as a drug substances of the type for which regulatory approval as a drug is required in for example the US or the European Union. Minerals and herbs may also be present in the oil phase. Such further components are used widely in the food, pharmaceutical and nutraceutical industries. The use of cellulose derivatives (e.g. hydroxy methyl propyl cellulose) as emulsion stabilizers is especially preferred. Examples of further substances which may be incorporated include materials such as niacin, amlodipine and ezetimibe which are currently co-formulated with statins, e.g. in the combined products marketed as Advicor®, Cadnet® and Vytorin®. These can be included at the relative dosages used in these commercial products.

The pH of the aqueous phase of the emulsion is preferably in the range 2 to 9, particularly 3 to 7.5.

The aqueous phase preferably has a gelling temperature in the range 10 to 30° C., more preferably 15 to 28° C., and a melting temperature in the range 20 to 80° C., more preferably 24 to 60° C., especially 28 to 50° C.

Where a sweetener is included in the aqueous phase, this will typically be selected from natural sweeteners such as sucrose, fructose, glucose, reduced glucose, maltose, xylitol, maltitol, sorbitol, mannitol, lactitol, isomalt, erythritol, polyglycitol, polyglucitol and glycerol and artificial sweeteners such as aspartame, acesulfame-K, neotame, saccharine, sucralose. The use of non-cariogenic sweeteners is preferred and the use of xylitol is especially preferred.

The statin may, if desired, be formulated together with a further solubility enhancing or amphiphilic agent so as to enhance its dissolution or dispersion within the aqueous, or more preferably the oil, phase of the emulsion.

Besides providing the statin and omega-3 to the patient in a readily consumable form, it is believed that the co-formulation of statin and omega-3 will enhance uptake of the statin from the gut.

Viewed from a further aspect the invention provides a method of treatment of a human subject to combat cardiovascular disease, said method comprising orally administering an effective amount of a composition according to the invention to said subject.

In an alternative aspect of the invention, the omega-3 acid ester and the statin may be in separate dose units, with the omega-3 acid ester presented as a gelled oil-in-water emulsion as described herein and the statin provided in any other separate dosage unit, e.g. a tablet, capsule, etc., especially in a conventional, commercially available form.

Viewed from this aspect the invention provides a method of treatment of a human subject to combat cardiovascular disease, which method comprises administering to said subject an effective amount of a statin and, in a separate dosage unit, an effective amount of a physiologically tolerable omega-3 acid ester, the improvement comprising administering said omega-3 acid ester in a dose unit comprising a gelled oil-in-water emulsion wherein the oil phase comprises said ester.

Viewed from a still further aspect the invention provides a physiologically tolerable gelled oil-in-water emulsion the oil phase whereof comprises a physiologically tolerable omega-3 acid ester for use in a method of treatment of a human subject to combat cardiovascular disease, which method comprises administering to said subject a statin and, in a separate dosage unit, said emulsion.

The dose units of the gelled emulsions of the compositions of the invention may be formed in conventional fashion, e.g. preparation of the emulsion and formation of the emulsion into a gelled mass for example by dosing into molds before gelation is complete or by cutting a gelled mass into individual dose units, and, if desired, coating the gelled dose units. Emulsification and subsequent steps involving unpackaged gel are preferably effected under a non-oxidizing atmosphere, e.g. a nitrogen atmosphere.

Particularly preferably the dose units are blister packed and accordingly it is especially desirable to use the blistered layer of the blister packaging as the mold. The blister pack can then be foil sealed. The use of oxygen-impermeable foil packaging is especially preferred, e.g. as both laminate of a blister pack or as a single dose unit containing sachet. Oxygen-impermeable foils, e.g. metal/plastics laminates, are well known in the food and pharmaceuticals industries.

Viewed from a further aspect the invention provides the use of a statin for the manufacture of a composition according to the invention for use in a method of treatment of a human.

Viewed from a still further aspect the invention provides a pharmaceutical package, preferably a blister pack or sachet, comprising a foil-encased composition according to the invention.

Embodiments of the invention will now be described in the following non-limiting examples and the accompanying drawings, in which:

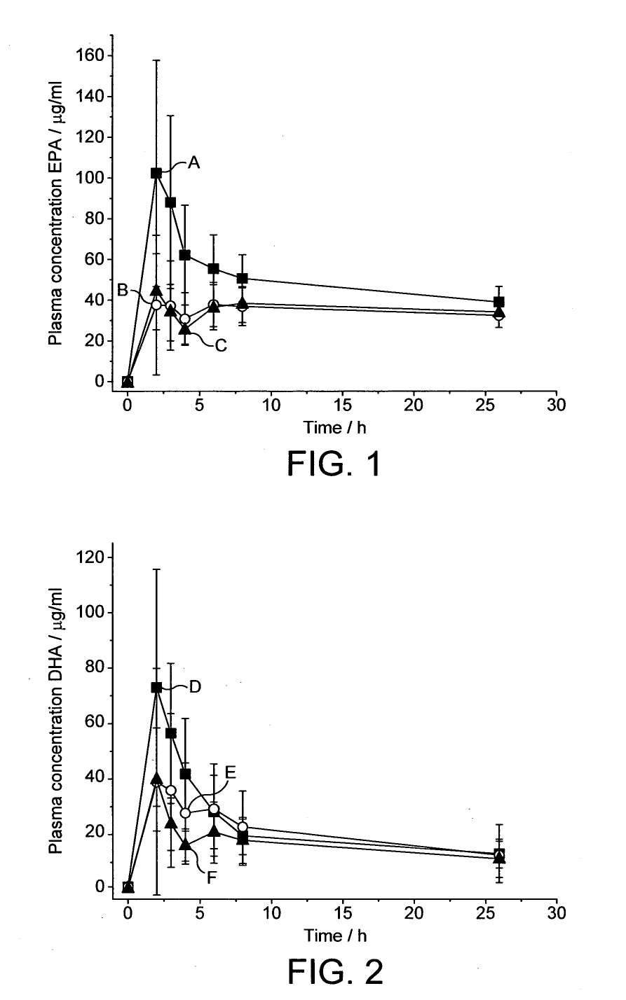

FIGS. 1 and 2 are graphs showing the omega-3 fatty acid concentration and composition of EPA and DHA respectively in total plasma delivered by three different administration forms;

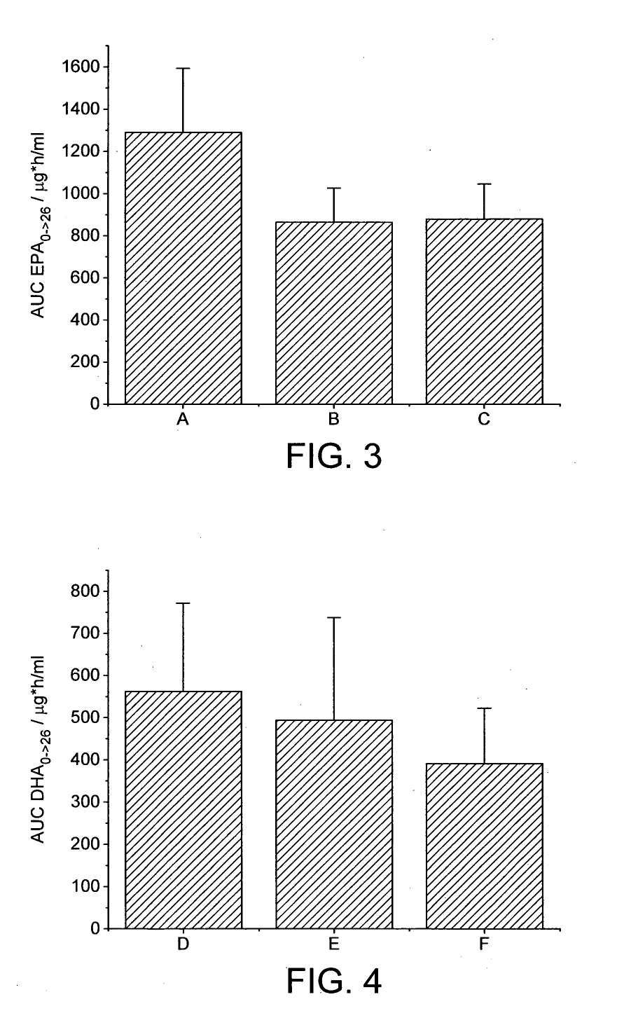

FIGS. 3 and 4 are graphs showing the total amount of EPA and DHA taken up respectively, i.e. the area under the curve of the graphs in FIGS. 1 and 2 respectively.

### EXAMPLE 1

### Statin-Free Composition

An aqueous phase is formed from the following ingredients:

| | | |
|---|---|---|
| Gelatin | 7.5% | wt |
| Xylitol | 36% | wt |
| Sorbitol | 14% | wt |
| 50% Citric acid | 1% | wt |
| Lemon flavour | 0.15% | wt |
| Water | ad 100% | wt |

Omega-3 ester (Omacor®) is emulsified with the aqueous phase in a weight ratio of 45:55 and the emulsion is poured in aliquots of 1.5 g into elongate moulds lined with a metal/plastics laminate blister tray and allowed to set. The blister tray is thermally sealed with a metal/plastics foil cover sheet.

### EXAMPLE 2

### Lovastatin-Containing Composition

Lovastatin is dissolved in the omega-3 ester used in Example 1 at concentrations of 30, 60 and 90 mg/g before emulsions are produced, poured and allowed to set as in Example 1. The set-gel dosage units are packaged as in Example 1.

Set-gel dosage units containing 5, 10, 20 and 40 mg rosuvastatin are produced analogously.

### EXAMPLE 3

### Fluvastatin-Containing Composition

Fluvastatin is dissolved in the aqueous phase used in Example 1 at concentrations of 24.5, 49, and 98 mg/g before emulsions are produced, poured and allowed to set as in Example 1. The set-gel dosage units are packaged as in Example 1.

Set-gel dosage units containing 10, 20, 40 and 80 mg pravastatin are produced analogously.

### EXAMPLE 4

### Gum Arabicum-Containing Compositions

An aqueous phase is prepared using the following components:

| | | |
|---|---|---|
| Gelatin | 5.7% | wt |
| Xylitol | 24.2% | wt |
| Sorbitol | 10.4% | wt |
| 50% Citric acid | 0.6% | wt |
| Lemon flavour | 1.1% | wt |
| Gum arabicum | 3.7% | wt |
| Water | ad 100% | wt |

Statin-free and statin-containing dose units are prepared using this aqueous phase analogously to Examples 1 to 3.

### EXAMPLE 5

### Atorvastatin-Containing Composition

An aqueous phase is prepared according to Example 1 but with an additional 1% wt hydroxypropyl methyl cellulose. This phase is divided into two parts. One part is processed as in Example 1 to form an emulsion. Atorvastatin is dissolved in sunflower oil at 44.4, 88.9, and 177.8 mg/g and using these solutions further emulsions are prepared as in Example 1 using the second part of the aqueous phase. The statin-containing emulsions are mixed with the EPA-ester emulsions in a weight ratio of 1:2 and poured into moulds and sealed as in Example 1.

### EXAMPLE 6

### Randomised, Controlled Trial

The absorption of omega-3 fatty acids delivered by two different administration forms (two different formulations of omega-3 food supplements) is compared.

5 g omega-3 fatty acids (2.805 g eicosapentaenoic acid (EPA), 1.87 g docosahexaenoic acid (DHA)) in triglyceride form and 13 mg Vitamin E were administered to students of 18-28 years of age, in the form of either a soft gelled oil-in-water emulsion or as standard softgel capsules. Blood samples were collected after 0, 2, 3, 4, 6, 8 and 26 hours. The fatty acid concentration and composition in total plasma were measured.

In FIGS. 1-4, A and D correspond to administration of the soft-gelled oil-in-water emulsion of the present invention containing EPA or DHA respectively, B and E correspond to the administration of a standard omega-3 soft gel capsules containing a liquid marine phospholipid core and C and F correspond to the administration of standard omega-3 soft gel capsules containing a liquid triglyceride core.

From FIGS. 1 and 2, it can be seen that lipophilic compounds (e.g. the omega-3 fatty acids EPA and DHA) are absorbed more quickly when administered in a soft gelled oil-in-water emulsion than when administered in the form of a standard soft gel capsule containing a liquid core.

From FIGS. 3 and 4, it can be seen that a higher total plasma concentration of lipophilic compounds (e.g. the omega-3 fatty acids EPA and DHA) is achieved when administered in a soft gelled oil-in-water emulsion than when administered in the form of a standard soft gel capsule containing a liquid core.

1. An oral pharmaceutical composition in unit dose form, each unit dose comprising a statin within a unitary carrier body, said body comprising a soft, chewable, gelled oil-in-water emulsion, one or both of the oil phase and the water phase whereof comprises a physiologically tolerable omega-3 acid ester.

2. The oral pharmaceutical composition of claim 1, wherein said physiologically tolerable omega-3 acid ester is an EPA ester, a DHA ester or a combination of EPA and DHA esters.

3. A composition as claimed in claim 1 or claim 2 wherein said statin is selected from lovastatin, simvastatin, pravastatin, atorvastatin, and rosuvastatin.

4. A composition as claimed in any one of claims 1 to 3 wherein said emulsion contains gelatin as a gelling agent.

5. A composition as claimed in any one of claims 1 to 4 consisting of a said gelled emulsion wherein the said statin is present in the aqueous phase.

6. A pharmaceutical package comprising a foil-encased composition as claimed in any one of claims 1 to 5.

7. A package as claimed in claim 6 in the form of a blister pack.

8. A process for preparing an oral pharmaceutical composition in unit dose form, which process comprises: forming an oil phase comprising a physiologically tolerable oil which comprises a physiologically tolerable omega-3 acid ester; forming an aqueous phase comprising an aqueous solution of a physiologically tolerable gelling agent; forming an oil-in-water emulsion with said oil phase and said aqueous phase; allowing said emulsion to gel to form a soft chewable mass; and, before, during or after gelling of said emulsion, dividing said emulsion into dose units; wherein a statin is incorporated within one or both of said oil and water phases.

9. The process of claim 8, wherein said statin is incorporated within the oil phase.

10. A process for preparing an oral pharmaceutical composition in unit dose form, which process comprises: forming an oil phase comprising a physiologically tolerable oil; forming an aqueous phase comprising an aqueous solution of a statin dissolved or dispersed therein; forming a water-in-oil emulsion with said oil phase and said aqueous phase, forming an oil-in-water emulsion with said water-in-oil phase and a further aqueous phase, comprising an aqueous solution of a physiologically tolerable gelling agent, allowing said emulsion to gel to form a soft chewable mass; and, before, during or after gelling of said emulsion, dividing said emulsion into dose units.

11. A method of treatment of a human subject to combat cardiovascular disease, said method comprising orally administering an effective amount of a composition as claimed in any one of claims 1 to 5 to said subject.

12. In a method of treatment of a human subject to combat cardiovascular disease, which method comprises administering to said subject an effective amount of a statin and, in a separate dosage unit, an effective amount of a physiologically tolerable omega-3 acid ester, the improvement comprising administering said omega-3 acid ester in a dose unit comprising a gelled oil-in-water emulsion wherein the oil phase comprises said ester.

13. A physiologically tolerable gelled oil-in-water emulsion the oil phase whereof comprises a physiologically tolerable omega-3 acid ester for use in a method of treatment of a human subject to combat cardiovascular disease, which method comprises administering to said subject a statin and, in a separate dosage unit, said emulsion.
